(19) United States
(12) Patent Application Publication
(10) Pub. No.: US 2003/0049308 A1
(43) Pub. Date: Mar. 13, 2003

(54) Transdermal or transmucosal dosage forms with a nicotine-containing active substance combination for smoker disintoxication

(76) Inventors: Frank Theobald, Bad Breisig (DE); Ulrich Frick, Pfalzfeld (DE)

Correspondence Address: BIRCH STEWART KOLASCH & BIRCH, PO BOX 747, FALLS CHURCH, VA, 22040-0747, US

(21) Appl. No.: 10/257,564

(22) PCT Filed: Apr. 2, 2001
(86) PCT No.: PCT/EP01/03712

(30) Foreign Application Priority Data
Apr. 15, 2000 (DE) 100 18 834.6

(51) Int. Cl. A61K 31/542; A61K 31/4439; A61K 9/70

(52) U.S. Cl. 424/449; 514/224.8; 514/343

(57) ABSTRACT

A transdermal or transmucosal pharmaceutical administration form for treating nicotine dependency or for disaccustoming smokers, comprising nicotine, a nicotine salt, a nicotine derivative or a nicotinergic substance in combination with at least one further active substance is characterized in that as additional active substance(s) it comprises at least one substance which acts on the central nervous system.

Transdermal or transmucosal administration forms with a nicotine-containing active substance combination for disaccustoming smokers.

The invention relates to transdermal or transmucosal pharmaceutical administration forms for treating nicotine dependency or for disaccustoming smokers, comprising nicotine, a nicotine salt, a nicotine derivative or a nicotinergic substance, in combination with a further active substance.

The invention further relates to the use of such administration forms for treating nicotine dependency, for the purpose of nicotine substitution, or for disaccustoming smokers, and also to the use of nicotine and/or its salts or derivatives for preparing transdermal or transmucosal pharmaceutical forms for treating nicotine dependency.

Although the harmful effects of smoking tobacco on health are common knowledge, ending the dependency is very difficult for the majority of those dependent on nicotine. The principal reason for this lies in the nicotine withdrawal symptoms which set in after the end of tobacco consumption by smoking. Withdrawal from this addictive dependency is therefore made easier if the nicotine demand is covered, at least during a disaccustomization phase, in another way, for example as part of a nicotine substitution therapy. This coverage may be provided, for example, by means of nicotine-containing transdermal therapeutic systems, known as nicotine patches. These patches are able to deliver nicotine through the skin to the human organism, thereby building up a plasma level which makes it possible to suppress the occurrence of nicotine withdrawal symptoms. As a result, smoker disaccustomization is made easier.

It has been found, however, that for many smokers the substance-related, i.e. nicotine-related, physical dependency is accompanied by a psychological dependency which cannot be treated by nicotine substitution alone. In many cases, the psychological dependency is responsible for the incidence of relapses.

In this connection it is worth mentioning that numerous clinical studies have shown that the combination of nicotine with an antidepressant, in particular, is able to improve the success rates in smoker disaccustomization. The success of a smoker disaccustomization therapy probably depends at least in part on the therapy of the psychological dependency. However, the supportive administration of psychopharmaceuticals is not without its problems, owing to the side-effect risk and the danger of over- and underdosing.

WO 96/00072 discloses transdermal and transmucosal pharmaceutical compositions which comprise nicotine in combination with caffeine or caffeine derivatives and which may be used for smoker disaccustomization. It is hypothesized that, in the case of smokers who are willing to give up, such a combination meets with improved acceptance. The aforementioned combination is additionally intended to prevent the weight increase which often occurs in the course of smoker disaccustomization. However, caffeine is unable to contribute toward overcoming the psychological dependency. Rather, there is a risk that the side-effects caused by caffeine, such as irritability, nervousness or muscular tremor, will increase the relapse rate.

It is an object of the present invention, therefore, to provide nicotine-containing pharmaceutical administration forms which at the same time permit the administration of an additional active substance, preferably an antidepressant, for combating the psychological dependency as part of a smoker disaccustomization therapy. In the context of the administration of this additional active substance, side-effects should be substantially ruled out, and it should be possible for the patients to use it simply and reliably.

Surprisingly, this object is achieved by means of a transdermal or transmucosal pharmaceutical administration form comprising nicotine, a nicotine salt, a nicotine derivative or a nicotinergic substance in combination with at least one further active substance, the formulation comprising as further active substance(s) at least one substance which acts on the central nervous system. Accordingly, the transdermal or transmucosal administration forms of the invention comprise a combination of the active substance nicotine, or a nicotine salt, a nicotine derivative or a nicotinergic substance, with at least one further substance which acts on the central nervous system.

With the aid of the administration forms of the invention it is possible on the one hand to administer to the patient the nicotine dose necessary for nicotine substitution, continuously over a defined period of time, and at the same time to administer a substance which acts on the central nervous system, for example an antidepressant or other psychopharmaceuticals, in order to suppress or combat the psychological dependency. It is particularly advantageous in this context that the administration takes place transdermally or else transmucosally and by means of a combination preparation. This avoids the patient having to take additional pharmaceuticals orally in order to combat his or her psychological dependency. It is therefore to be assumed that the patient compliance is improved by the administration of a single pharmaceutical comprising an active substance combination.

The administration of a substance which acts on the central nervous system via the transdermal (or else transmucosal) route makes it possible, moreover, to establish an effective blood plasma level which can be maintained over the entire application period. The reason why this is significant is that, especially in the case of centrally acting substances, the success of a therapy depends to a large extent on the presence of a constant plasma level. By means of the transdermal or transmucosal administration of the invention, the incidence of unwanted side-effects—as a consequence of exceeding or falling below the therapeutically effective dose—can be very largely prevented. Moreover, it also substantially rules out the misuse of the active substances, e.g. psychopharmaceuticals, that are present therein.

Additionally, the administration forms of the invention permit the continuous, transdermal or transmucosal administration of nicotine or nicotine derivatives, so that it is possible to build up and maintain a nicotine blood plasma level which is suitable for nicotine substitution as part of a smoker disaccustomization therapy.

The target nicotine blood level is in the range from 1 ng/ml to about 100 ng/ml, preferably from 10 ng/ml to 70 ng/ml, with particular preference between 10 and 30 ng/ml. It should be borne in mind that these values may vary in the case of individual patients. The skilled worker is aware of the means by which he or she can control the release characteristics of transdermal or transmucosal administration forms in order to allow the desired values.

It is preferred in this context for the desired plasma level to be reached after no later than 1 h, preferably after no later than 30 min following application.

The substance which acts on the central nervous system and is present in the administration forms of the invention in addition to nicotine preferably comprises an active substance from the group of psychopharmaceuticals which embraces the active-substance groups of the antidepressants, tranquillizers, nootropics, neuroleptics, psychotonics and psychomimetics. Particular preference is given here to active substances from the group of the antidepressants, since they have been found to be very suitable in respect of overcoming the psychological dependency. The invention additionally embraces nicotine-containing administration forms of the aforementioned kind which comprise, as additional active substances, two or more psychopharmaceuticals from the aforementioned active-substance groups.

The additional substance which acts on the central nervous system may be selected in particular from the group which embraces the active-substance groups of the phenothiazines, azaphenothiazines, thioxanthenes, butyrophenones, diphenylbutylpiperidines, iminodibenzyl derivatives, iminostilbene derivatives, dibenzocycloheptadiene derivatives, dibenzodiazepine derivatives, dibenzoxepin derivatives, benzodiazepines, indole derivatives, phenylethylamine derivatives and hypericin derivatives.

Furthermore, the invention includes in particular those nicotine-containing, transdermal or transmucosal administration forms which comprise as additional active substance a substance which acts on the central nervous system and which is selected from the group that contains the active substances chlorpromazine, perphenazine, sulpiride, clozapine, clomipramine, trimipramine, desipramine, imipramine, doxepin, risperidone, reserpine, maprotiline, mianserin, lofepramine, tranylcypromine, moclobemide, amitriptyline, paroxetine, promethazine, flupentixol, oxitriptan, viloxazine, meprobamate, hydroxyzine, buspirone, fenetylline, methylphenidate, prolintane, fenfluramine, fluvoxamine, meclofenoxate, nicergoline, piracetam, pyritinol, amfebutamone, and also salts and derivatives of these compounds.

Nicotine salts and nicotine derivatives which may be used in the administration forms of the invention are preferably nicotine hydrochloride, nicotine dihydrochloride, nicotine sulphate, nicotine bitartrate, nicotine-zinc chloride and nicotine salicylate, either individually or in combination, or else in combination with nicotine.

As substances with a nicotinergic effect, i.e. substances with an effect on the nicotine receptor, it is preferred to use—besides nicotine itself—lobeline, succinylcholine and other peripheral muscle relaxants.

The active substance doses and plasma levels that are suitable for treating the psychological dependency are known to the skilled worker. Preferably, the dose of the substance that acts on the central nervous system is tailored to the nicotine dose that it present in the administration form in such a way that both active substances as far as possible build up the particular therapeutically favourable plasma level and ensure a uniformly continuing active-substance release over a predeterminable period of application.

In one group of embodiments of the invention, the transdermal administration forms are in the form of transdermal therapeutic systems (TTS) which can be bonded as active substance patches to the skin of the patient where they subsequently release the active substances via the skin. The structure of such a system essentially comprises an active-substance-impermeable backing layer, a preferably pressure-sensitively adhering active substance reservoir connected to the backing layer, and a protective layer which can be removed prior to application. The active substance reservoir contains a combination of at least two active substances, namely nicotine, a nicotine salt, a nicotine derivative or a nicotinergic substance, and as further active substance component(s) additionally at least one substance which acts on the central nervous system and which may be selected from the substances or groups of substances mentioned above.

The active substance reservoir of the TTS, containing the active substances in dissolved or dispersed form, is built up substantially on the basis of polymers which form an active substance matrix; these polymers are also referred to as base polymers. The active substance reservoir preferably possesses pressure-sensitive adhesive properties which are governed by the nature of the base polymers used or by additionally admixed tackifying substances.

Furthermore, it is also possible to provide the active substance reservoir on the skin side with a pressure-sensitively adhering layer. In its simplest embodiment, the active substance reservoir of the TTS of the invention is a single-layer polymer matrix. Deviating from this, the TTS may feature a layer-form structure of the active substance reservoir, with at least two polymer matrix layers. In this case it may be advantageous if two polymer layers differ in their active substance content. For example, one of the active substance matrix layers of the active substance reservoir may contain nicotine as active substance and a second matrix layer may contain an antidepressant as the second active substance provided in accordance with the invention.

As the base polymers of the TTS of the invention it is preferred in particular to use acrylate copolymers, and also rubber/resin mixtures, synthetic rubbers, polyvinyl acetate, polyvinylpyrrolidones, silicone polymers, cellulose derivatives, hot-melt adhesives, and many other materials whose use on the human skin is unobjectionable. It is also possible to use mixtures of different base polymers.

The active substance polymer matrix may further comprise auxiliaries and additives, especially additives which promote skin permeation and which are known to the skilled worker.

In addition to the active substance reservoir discussed above, the structure of the TTS of the invention comprises a backing layer, which is impermeable to active substance, and a removable protective film, which is likewise impermeable to active substance.

Suitable materials for the backing layer include a large number of skin-compatible polymer films, such as films of polyvinyl chloride, ethylene vinyl acetate, vinyl acetate, polyethylene, polypropylene or cellulose derivatives, for example. Particularly suitable materials for the backing layer are polyesters, which are notable for particular strength. In certain cases it may also be useful to provide the film material backing layer with an additional applied layer: for example, by vapour deposition with metals or other diffusion barrier additives such as silicon dioxide, aluminium oxide or similar substances known to the skilled worker.

For the removable protective layer of the TTS it is possible in principle to use the same materials as for the backing layer, subject to the proviso that this layer is subjected to a suitable surface treatment, e.g. fluorosiliconization, so that it can be removed from the pressure-sensitive adhesive layer it covers and can be removed before the TTS is applied. Furthermore, other materials as well can be used as removable protective layers, such as polytetrafluoroethylene-treated paper, cellophane, polyvinyl chloride or the like, for example.

The above-described TTS may be produced using processes which are known to the skilled worker. Generally, a solution or melt of the matrix base polymers is prepared in which the active substances are dissolved or homogeneously dispersed. Subsequently, the composition containing active substances is coated onto a substrate in film form, dried, and covered with a further film layer. Individual active substance patches are then punched from the resulting laminate. The area of the individual patches is preferably dimensioned such that one patch corresponds in each case to a half-daily dose or a whole daily dose or a 2-day or 3-day dose, based in each case on nicotine and/or the combination active substance (e.g. antidepressant).

In a further group of embodiments of the invention, the administration forms comprising nicotine and at least one substance which acts on the central nervous system are designed as transmucosal administration forms. Transmucosal administration forms of this kind may be employed, for example, in the oral cavity for sublingual or buccal administration. In this case the active substance is absorbed by way of the mucosa, bypassing the digestive tract.

A transmucosal administration form of the invention comprising a combination of nicotine with one of the abovementioned active substances is preferably designed as a flat, film-like, foil-like or wafer-like active substance carrier and is provided with mucoadhesive properties. This ensures the adhesion of the pharmaceutical form to the surface during the period of application. The mucoadhesive properties may be brought about through the addition of suitable polymeric auxiliaries, which preferably have water-swellable properties, examples being starch, carboxymethylcellulose, hydroxypropylcellulose, polyacrylic acid, polyvinylpyrrolidones, polyethylene oxide polymers, ethylcellulose or propylcellulose, alginates, pectins or natural gums.

The administration forms of the invention combine the advantages of a combination therapy, comprising nicotine or a nicotine salt, a nicotine derivative or a nicotinergic substance, for substitution therapy, and a substance which acts on the central nervous system, for the treatment of the psychological dependency, with the advantages of transdermal or transmucosal administration, respectively. For these reasons, they may be used with advantage to treat nicotine dependency, for disaccustoming smokers, and for nicotine substitution. Unlike known active substance plasters containing nicotine, the administration forms of the invention not only permit nicotine substitution but at the same time allow treatment of the psychological dependency component of nicotine addiction.

The administration forms of the invention are preferably used to administer nicotine, or a substance related to nicotine, in combination with an appropriate antidepressant or another substance which is suitable for treating the psychological dependency and which acts on the central nervous system to patients via the transdermal or transmucosal route as part of a treatment for disaccustoming smokers.

1. Transdermal or transmucosal pharmaceutical administration form for treating nicotine dependency or for disaccustoming smokers, comprising nicotine, a nicotine salt, a nicotine derivative or a nicotinergic substance in combination with at least one further active substance, characterized in that as additional active substance(s) it comprises at least one substance which acts on the central nervous system, said additional substance which acts on the central nervous system being selected from the group of the psychopharmaceuticals which embraces the active-substance groups of the antidepressants, tranquilizers, nootropics, neuroleptics, or psychomimetics.

2. Administration form according to claim 1, characterized in that it comprises a nicotine derivative or a nicotine salt which is preferably selected from the group which encompasses nicotine hydrochloride, nicotine dihydrochloride, nicotine sulphate, nicotine bitartrate, nicotine-zinc chloride and nicotine salicylate.

3. Administration form according to claim 1, characterized in that it comprises a nicotinergic substance selected preferably from the group encompassing nicotine, lobeline, succinylcholine and other peripheral muscle relaxants.

4. Administration form according to any one of the preceding claims, characterized in that the additional substance which acts on the central nervous system is selected from the group which embraces the active-substance groups of the phenothiazines, azaphenothiazines, thioxanthenes, butyrophenones, diphenylbutylpiperidines, iminodibenzyl derivatives, iminostilbene derivatives, dibenzocycloheptadiene derivatives, dibenzodiazepine derivatives, dibenzoxepin derivatives, benzodiazepines, indole derivatives, phenylethylamine derivatives and hypericin derivatives.

5. Administration form according to any one of the preceding claims, characterized in that the additional substance which acts on the central nervous system is selected from the group containing the active substances chlorpromazine, perphenazine, sulpiride, clozapine, clomipramine, trimipramine, desipramine, imipramine, doxepin, risperidone, reserpine, maprotiline, mianserin, lofepramine, tranylcypromine, moclobemide, amitriptyline, paroxetine, promethazine, flupentixol, oxitriptan, viloxazine, meprobamate, hydroxyzine, buspirone, fenetylline, methylphenidate, prolintane, fenfluramine, fluvoxamine, meclofenoxate, nicergoline, piracetam, pyritinol, amfebutamone, and also salts and derivatives of these compounds.

6. Administration form according to one or more of the preceding claims, characterized in that it permits the transdermal administration of the active substances, the administration form preferably being designed as a transdermal therapeutic system which comprises a backing layer which is impermeable to active substance, a preferably pressure-sensitively adhering active substance reservoir containing nicotine, a nicotine salt, a nicotine derivative or a nicotinergic substance and also, in addition, at least one substance which acts on the central nervous system, and a protective layer which can be removed prior to application.

7. Administration form according to one or more of the preceding claims, characterized in that it permits the transmucosal administration of the active substances, the administration form preferably being designed as a flat, film-like, foil-like or wafer-like active substance carrier and being provided with mucoadhesive properties, the mucoadhesive properties being brought about by the addition of a polymeric auxiliary, preferably from the group encompassing starch, carboxymethylcellulose and polyacrylic acid.

8. Use of a transdermal or transmucosal administration form for the transdermal or transmucosal administration of nicotine, a nicotine salt, a nicotine derivative or a nicotinergic substance in combination with at least one substance which acts on the central nervous system for the treatment of nicotine dependency, for nicotine substitution, or for smoker disaccustomization, with the substance which acts on the central nervous system being selected from the group of the psychopharmaceuticals which encompasses the active-substance groups of the antidepressants, tranquillizers, nootropics, neuroleptics, and psychomimetics.

9. Use according to claim 8, characterized in that the substance which acts on the central nervous system is selected from the group which embraces the active-substance groups of the phenothiazines, azaphenothiazines, thioxanthenes, butyrophenones, diphenylbutylpiperidines, iminodibenzyl derivatives, iminostilbene derivatives, dibenzocycloheptadiene derivatives, dibenzodiazepine derivatives, dibenzoxepin derivatives, benzodiazepines, indole derivatives, phenylethylamine derivatives and hypericin derivatives.

10. Use according to claim 8, characterized in that the substance which acts on the central nervous system is selected from the group containing the active substances chlorpromazine, perphenazine, sulpiride, clozapine, clomipramine, trimipramine, desipramine, imipramine, doxepin, risperidone, reserpine, maprotiline, mianserin, lofepramine, tranylcypromine, moclobemide, amitriptyline, paroxetine, promethazine, flupentixol, oxitriptan, viloxazine, meprobamate, hydroxyzine, buspirone, fenetylline, methylphenidate, prolintane, fenfluramine, fluvoxamine, meclofenoxate, nicergoline, piracetam, pyritinol, amfebutamone, and also salts and derivatives of these compounds.

11. Use of nicotine, a nicotine salt, a nicotine derivative or a nicotinergic substance in combination with at least one substance which acts on the central nervous system and is selected from the group of the psycho-pharmaceuticals which encompasses the active-substance groups of the antidepressants, tranquillizers, nootropics, neuroleptics, and psychomimetics, for producing a transdermal therapeutic system or a transmucosal administration form for the treatment of nicotine dependency, for smoker disaccustomization, or for nicotine substitution.

12. Use according to claim 11, characterized in that the substance which acts on the central nervous system is selected from the group which embraces the active-substance groups of the phenothiazines, azaphenothiazines, thioxanthenes, butyrophenones, diphenylbutylpiperidines, iminodibenzyl derivatives, iminostilbene derivatives, dibenzocycloheptadiene derivatives, dibenzodiazepine derivatives, dibenzoxepin derivatives, benzodiazepines, indole derivatives, phenylethylamine derivatives and hypericin derivatives.

13. Use according to claim 11, characterized in that the substance which acts on the central nervous system is selected from the group containing the active substances chlorpromazine, perphenazine, sulpiride, clozapine, clomipramine, trimipramine, desipramine, imipramine, doxepin, risperidone, reserpine, maprotiline, mianserin, lofepramine, tranylcypromine, moclobemide, amitriptyline, paroxetine, promethazine, flupentixol, oxitriptan, viloxazine, meprobamate, hydroxyzine, buspirone, fenetylline, methylphenidate, prolintane, fenfluramine, fluvoxamine, meclofenoxate, nicergoline, piracetam, pyritinol, amfebutamone, and also salts and derivatives of these compounds.
